SEQUENCE LISTING (25 sequences)

SEQ ID NO: 1 - protein, 757 aa, synthetic construct
SSGGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVGVPWP

SEQ ID NO: 2 - protein, 112 aa, Methylobacterium extorquens
APTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIR

SEQ ID NO: 3 - protein, 16 aa, synthetic construct
GSGSGSGSGSGSGSGS

SEQ ID NO: 4 - protein, 892 aa, synthetic construct
MHHHHHHSSGGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVGVPWPGSGSGSGSGSGSGSGSAPTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIR

SEQ ID NO: 5 - DNA, 2679 bp, synthetic construct
  source 1..2679: mol_type = other DNA
atgcatcatcatcatcatcacagttcagggggtgtaggtgtccctggagtaggcgttccaggcgtcggggtacctggcgtgggtgttcccggcgttggagtcccgggtgtaggagtccccggtgttggtgtacctggagtgggagtaccgggagtaggagtaccaggagtgggggttcctggtgtgggagtcccaggagtcggtgtaccaggggtaggggtaccgggcgtaggtgtgccaggagttggggtgcctggagtcggagtccctggtgtcggagtacctggtgttggagtacccggagttggtgttcctggagttggagttccgggggtaggagttccaggagtaggggtcccaggtgttggcgtaccaggtgtgggcgtgcccggggtcggcgtccctggggtaggtgttccaggtgtaggggttccaggggtgggagttcctggggttggagtgcctggtgtaggtgtcccaggagtaggtgtccctggagtaggcgttccaggcgtcggggtacctggcgtgggtgttcccggcgttggagtcccgggtgtaggagtccccggtgttggtgtacctggagtgggagtaccgggagtaggagtaccaggagtgggggttcctggtgtgggagtcccaggagtcggtgtaccaggggtaggggtaccgggcgtaggtgtgccaggagttggggtgcctggagtcggagtccctggtgtcggagtacctggtgttggagtacccggagttggtgttcctggagttggagttccgggggtaggagttccaggagtaggggtcccaggtgttggcgtaccaggtgtgggcgtgcccggggtcggcgtccctggggtaggtgttccaggtgtaggggttccaggggtgggagttcctggggttggagtgcctggtgtaggtgttccaggtgtaggtgtccctggagtaggcgttccaggcgtcggggtacctggcgtgggtgttcccggcgttggagtcccgggtgtaggagtccccggtgttggtgtacctggagtgggagtaccgggagtaggagtaccaggagtgggggttcctggtgtgggagtcccaggagtcggtgtaccaggggtaggggtaccgggcgtaggtgtgccaggagttggggtgcctggagtcggagtccctggtgtcggagtacctggtgttggagtacccggagttggtgttcctggagttggagttccgggggtaggagttccaggagtaggggtcccaggtgttggcgtaccaggtgtgggcgtgcccggggtcggcgtccctggggtaggtgttccaggtgtaggggttccaggggtgggagttcctggggttggagtgcctggtgtaggtgtgccgggagtaggtgtccctggagtaggcgttccaggcgtcggggtacctggcgtgggtgttcccggcgttggagtcccgggtgtaggagtccccggtgttggtgtacctggagtgggagtaccgggagtaggagtaccaggagtgggggttcctggtgtgggagtcccaggagtcggtgtaccaggggtaggggtaccgggcgtaggtgtgccaggagttggggtgcctggagtcggagtccctggtgtcggagtacctggtgttggagtacccggagttggtgttcctggagttggagttccgggggtaggagttccaggagtaggggtcccaggtgttggcgtaccaggtgtgggcgtgcccggggtcggcgtccctggggtaggtgttccaggtgtaggggttccaggggtgggagttcctggggttggagtgcctggtgtaggtgttcctggggtaggtgtccctggagtaggcgttccaggcgtcggggtacctggcgtgggtgttcccggcgttggagtcccgggtgtaggagtccccggtgttggtgtacctggagtgggagtaccgggagtaggagtaccaggagtgggggttcctggtgtgggagtcccaggagtcggtgtaccaggggtaggggtaccgggcgtaggtgtgccaggagttggggtgcctggagtcggagtccctggtgtcggagtacctggtgttggagtacccggagttggtgttcctggagttggagttccgggggtaggagttccaggagtaggggtcccaggtgttggcgtaccaggtgtgggcgtgcccggggtcggcgtccctggggtaggtgttccaggtgtaggggttccaggggtgggagttcctggggttggagtgcctggtgtaggtgttggagttccctggccgggatccggctctggttctggttctggtagcggttctggctctggttctgctccaaccacgaccacaaaggtcgacattgccgcttttgaccccgataaagatggaacgatcgatctgaaggaagcacttgctgccggatcagccgcgttcgacaagctggacccagataaggacggaaccctggacgcgaaagaactgaaaggtcgtgtctcagaggcagacttaaaaaaacttgatcctgacaatgacggaaccttagataaaaaggaataccttgccgctgtcgaagcacaatttaaagcggcgaatccagataatgacggaacgattgatgctcgtgaattggcctcacctgcggggtccgcgttggtcaacttaattcgttaa

SEQ ID NO: 6 - DNA, 69 bp, synthetic construct
  source 1..69: mol_type = other DNA
gatccggctctggttctggttctggtagcggttctggctctggttctgctccaaccacgaccacaaagg

SEQ ID NO: 7 - DNA, 69 bp, synthetic construct
  source 1..69: mol_type = other DNA
tcgacctttgtggtcgtggttggagcagaaccagagccagaaccgctaccagaaccagaaccagagccg

SEQ ID NO: 8 - protein, 21 aa, Methylobacterium extorquens
MAFRLSSAVLLAALVAAPAYA

SEQ ID NO: 9 - protein, 133 aa, Methylobacterium extorquens
MAFRLSSAVLLAALVAAPAYAAPTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIR

SEQ ID NO: 10 - protein, 764 aa, synthetic construct
MHHHHHHSSGGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVGVPWP

SEQ ID NO: 11 - protein, 899 aa, synthetic construct
MHHHHHHSSGGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVGVPWPGSMAFRLSSAVLLAALVAAPAYAAPTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIR

SEQ ID NO: 12 - DNA, 399 bp, Methylobacterium extorquens
  source 1..399: mol_type = genomic DNA
atggcgtttcgcctttcaagtgccgtcctgttagctgcccttgttgccgcaccagcatacgcggctccaaccacgaccacaaaggtcgacattgccgcttttgaccccgataaagatggaacgatcgatctgaaggaagcacttgctgccggatcagccgcgttcgacaagctggacccagataaggacggaaccctggacgcgaaagaactgaaaggtcgtgtctcagaggcagacttaaaaaaacttgatcctgacaatgacggaaccttagataaaaaggaataccttgccgctgtcgaagcacaatttaaagcggcgaatccagataatgacggaacgattgatgctcgtgaattggcctcacctgcggggtccgcgttggtcaacttaattcgt

SEQ ID NO: 13 - protein, 14 aa, synthetic construct
GSGSGSGSGSGSGS

SEQ ID NO: 14 - protein, 5 aa, synthetic construct
GGGGS

SEQ ID NO: 15 - protein, 6 aa, synthetic construct
GSGSGS

SEQ ID NO: 16 - protein, 5 aa, synthetic construct
GGSGG

SEQ ID NO: 17 - protein, 8 aa, synthetic construct
GGSGGGGG

SEQ ID NO: 18 - protein, 11 aa, synthetic construct
GGSGGSGGSGG

SEQ ID NO: 19 - protein, 5 aa, synthetic construct
VPGXG

SEQ ID NO: 20 - protein, 5 aa, synthetic construct
VPAXG

SEQ ID NO: 21 - protein, 5 aa, synthetic construct
VPGVG

SEQ ID NO: 22 - protein, 6 aa, synthetic construct
HHHHHH

SEQ ID NO: 23 - protein, 7 aa, synthetic construct
HHHHHHH

SEQ ID NO: 24 - protein, 8 aa, synthetic construct
HHHHHHHH

SEQ ID NO: 25 - protein, 5 aa, synthetic construct
GVGVP

(19) United States
(12) Patent Application Publication
(10) Pub. No.: US 2024/0352092 A1
(43) Pub. Date: Oct. 24, 2024

(54) PEPTIDE, COMPOSITION FOR RECOVERING RARE EARTH ELEMENTS COMPRISING SAME, AND RECOVERY METHOD

(71) Applicant: GWANGJU INSTITUTE OF SCIENCE AND TECHNOLOGY, Gwangju (KR)

(72) Inventors: INCHAN KWON, Gwangju (KR); ZOHAIB HUSSAIN, Gwangju (KR); SEOUNG KYUN KIM, Gwangju (KR)

(21) Appl. No.: 18/580,752

(22) PCT Filed: Dec. 14, 2022
(86) PCT No.: PCT/KR2022/020368

§ 371 Date: Jan. 19, 2024

(30) Foreign Application Priority Data
Dec. 14, 2021 (KR) 10-2021-0179119
Dec. 14, 2022 (KR) 10-2022-0174913

(51) Int. Cl. C07K 14/78; B01D 21/26

(52) U.S. Cl. CPC C07K 14/78; B01D 21/262

(57) ABSTRACT

A peptide has the sequence of X-(L)n-Y, wherein one of X and Y is an amino acid sequence of an elastin-like polypeptide, the other is an amino acid sequence of SEQ ID NO: 2, L is a linker sequence, and n is 0 or 1. By using the peptide, rare earth elements can be recovered with high yield from a sample including the rare earth elements. In addition, the peptide can be used repeatedly, and can also minimize environmental pollution problems since the peptide corresponds to a biosorbent, and thus has very high industrial applicability.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2022/020368 filed on Dec. 14, 2022, which claims priority to the benefit of Korean Patent Application Nos. 10-2021-0179119 filed on Dec. 14, 2021 and 10-2022-0174913 filed on Dec. 14, 2022, in the Korean Intellectual Property Office, the entire contents of which are incorporated herein by reference.

### BACKGROUND

### 1. Technical Field

The present invention relates to a novel peptide, a composition for recovering rare earth elements including the same, and a recovery method of the rare earth elements.

### 2. Background Art

Rare earth elements (REEs) are applied to various fields such as catalysts, ceramics, phosphors, and alloys due to their unique electronic and magnetic properties. Global demand for REE has grown steadily in recent decades due to its importance in the consumer electronics industry and clean energy fields. However, excessive REE mining due to the increased demand for such REE may cause environmental problems because it increases environmental pollution in soil, water and plants. Various REE recovery methods are being studied to reduce the contamination and use of REE. Most of the pyrometallurgical and hydrometallurgical techniques available for REE extraction and recovery from the above dilute sources are technically difficult, complicated, expensive, and not environmentally friendly. Therefore, in order to solve the problem of REE supply, it is important to develop selective, efficient and environmentally friendly methods for REE recovery.

Biosorption has been extensively studied as an environmentally friendly and cost-effective method for recovering REE from aqueous solutions. For example, there are proteins engineered to convert a target metal to REE, but these engineered proteins are still bound to metals other than REE. Therefore, these proteins have limitations in selectively recovering REE from a mixture of REE and non-REE. Another example of a biosorbent is natural microbial cells, which have several disadvantages such as fouling, limited operating conditions and selectivity. Further, repeated recovery of REE by the microbial cell-based biosorbent is more difficult since harsh conditions are required and it is not easy to separate the REE desorbed from the protein-based biosorbent.

Accordingly, it is necessary to develop a biosorbent that can overcome the limitation of difficulty in repeated use of biosorbents for REE recovery.

### SUMMARY

An object of the present invention is to provide a novel peptide with excellent recovery performance of rare earth elements.

Another object of the present invention is to provide a composition for recovering rare earth elements, which includes the novel peptide capable of recovering rare earth elements in high yield and enabling repeated use, as well as a recovery method of the rare earth elements.
1. A peptide including the following sequence:

X-(L)n-Y
(wherein one of X and Y is an amino acid sequence of an elastin-like polypeptide, the other is an amino acid sequence of SEQ ID NO: 2, L is a linker sequence, and n is 0 or 1).
2. The peptide according to the above 1, wherein the elastin-like polypeptide consists of the amino acid sequence of SEQ ID NO: 1.
3. The peptide according to the above 1, wherein L is GS or an amino acid sequence of SEQ ID NO: 3.
4. A composition for recovering rare earth elements, including the peptide according to any one of the above 1 to 3.
5. A method for recovering rare earth elements, including adding the peptide according to any one of the above 1 to 3 to a sample including rare earth elements to adsorb the rare earth elements to the peptide.
6. The method according to the above 5, wherein the step of adsorbing the rare earth element to the peptide is performed at a temperature less than a transition temperature of the peptide and a pH greater than 3 to 6.
7. The method according to the above 5, further including: adjusting the sample added with the peptide to a temperature higher than the transition temperature of the peptide to form aggregates of the peptide to which the rare earth elements are adsorbed;
centrifuging the sample to obtain pellets including aggregates of the peptide to which the rare earth elements are adsorbed;
adjusting the pellets to a temperature less than the transition temperature of the peptide and a pH of less than 3 thus to solubilize the aggregates of the peptide adsorbed with the rare earth elements and desorb the rare earth elements from the peptide;
forming the aggregates of the peptide by adjusting the mixture including the desorbed rare earth elements and the solubilized peptide to a temperature higher than the transition temperature of the peptide; and
recovering the desorbed rare earth elements from the supernatant isolated and obtained by centrifuging the mixture.
8. The method according to the above 7, further including: recovering the aggregates of the peptide from the pellets obtained by centrifuging the mixture; and
adjusting the recovered aggregates of the peptide to a temperature less than the transition temperature of the peptide thus to solubilize the aggregates.

The present invention provides a novel peptide capable of recovering rare earth elements, such that the rare earth elements can be recovered in high yield from a sample including the rare earth elements using the above peptide. Further, the peptide can be used repeatedly and correspond to a biosorbent to minimize a problem of environmental pollution, and thereby achieving very high industrial applicable value.

### BRIEF DESCRIPTION OF THE DRAWINGS

FIG. 1 is a schematic diagram of a method for recovering rare earth elements (REEs) using RELP (REEs-sensitive and thermo-responsive genetically encoded ELP) of the present embodiment (Step 1: Selective adsorption of REE by RELP from a metal mixture at 4° C. and pH 5.8, Step 2: Coacervation of RELP at 37° C., followed by centrifugation at 37° C. to obtain non-rare earth elements (non-REEs) in the supernatant, Step 3: Solubilization of coacervate and desorption of bound REEs in a desorption buffer (pH 2.2) at 4° C., Step 4: Coacervation of RELP by adding 0.3 M NaCl at 37° C., followed by recovery of coacervate and REE through centrifugation at 37° C., Step 5: Solubilization of RELP coacervate at 4° C. and pH 5.8).

FIGS. 2A to 2F show purification and phase transition behavior of elastin-like polypeptide (ELP) and RELP. Wherein FIG. 2A illustrates results of confirming the purified ELP and RELP by SDS-PAGE analysis, FIG. 2B illustrates a turbidity profile for the purified RELP with inverse-phase transition behavior, FIG. 2C illustrates results of visually confirming the inverse-phase transition by RELP during cooling and heating, FIGS. 2D and 2E show the size distributions of ELP and RELP at a temperature (37° C.) higher than the transition temperature (Tₜ) by DLS analysis, respectively, and FIG. 2F shows the reversible size change indicating the reversible phase transition behavior of ELP and RELP at Tₜ (10° C.) or lower and Tₜ (37° C.) or higher.

FIG. 3 shows results of titrating XO with each REE (Y³⁺, La³⁺, Ce³⁺, or Tb³⁺) in the absence or presence of RELP, wherein REE-XO refers to a case without RELP, and RELP-REE-XO refers to a case with RELP.

FIGS. 4A and 4B show results of titrating XO with Cu²⁺ (FIG. 4A) and Zn²⁺ (FIG. 4B) in the absence or presence of RELP, wherein the concentrations of RELP and XO were 3.0 and 7 μM, respectively.

FIG. 5 shows results of titrating XO with Tb³⁺, wherein Tb—XO refers to the case without RELP and Ca²⁺ (Tb—XO), RELP-Tb—XO refers to the case with only 5.0 μM RELP, and RELP-Tb:Ca—XO refers to the case with 5.0 μM RELP and equivalent of Ca²⁺ (2-20 μM).

FIG. 6 shows the stoichiometry of REE binding to RELP monitored by competitive titration of RELP in the presence of XO (7 μM).

FIG. 7 shows results of confirming an effect of pH on Tb³⁺ recovery by RELP, wherein the concentration of RELP was 100 μM, and the recovery yield of Tb³⁺ was represented according to pH.

FIG. 8A shows a binding ratio of 100 μM mixed REE (the concentration of each REE is about 7.7 μM) by 100 μM RELP in the presence of competing non-REE metals, wherein metal concentrations were measured by ICP-MS. FIG. 8B shows a total binding ratio of the mixed REE by RELP compared to non-REE.

FIG. 9A shows selective recovery of the mixed REE by RELP in the presence of an excess of competing non-REE metals, wherein metal concentrations were measured by ICP-MS. FIG. 9B shows a total recovery rate of the mixed REE by RELP compared to non-REE.

FIGS. 10A and 10B show reusability of RELP for REE recovery. Specifically, FIG. 10A shows the calculated recovery capacity for each successive recovery cycle per RELP, wherein the same amount of protein ([RELP]=75 μM) was used for 6 consecutive cycles, and each cycle consists of an adsorption step with 200 μM Tb³⁺ (pH 5.8) and a desorption step (pH 2.2). Further, FIG. 10B shows the recovery efficiency of RELP for 8 consecutive cycles using the same amount of protein ([RELP]=100 μM), wherein each cycle consists of an adsorption step and a desorption step (pH 2.2) of 50 μM Tb³⁺ (pH 5.8) (Adsorption buffer: 20 mM MES buffer. Desorption buffer: 4 mM Na₂HPO₄, 100 mM citric acid. Metal concentrations were measured by ICP-MS).

FIG. 11 shows results of recovering REE from steel slag leachate using RELP (pH=5.6. [RELP]=50 μM), wherein the recovery yield was calculated as the amount of metal recovered relative to the initial amount added, and the initial metal concentration of the steel slag leachate is described in Table 3 to be described below.

FIG. 12 shows a schematic diagram of RELP purification by inverse-transition cycling (ITC).

FIG. 13 shows a surface displacement analysis based on xylenol orange (XO) and RELP competing for selected metals.

### DETAILED DESCRIPTION

Hereinafter, the present invention will be described in detail.

The present invention provides a novel peptide.

The peptide of the present invention consist of the following sequence:

X-(L)n-Y
(wherein one of X and Y is an amino acid sequence of an elastin-like polypeptide, the other is an amino acid sequence of SEQ ID NO: 2, L is a linker sequence, and n is 0 or 1).

With regard to the peptide of the present invention, an elastin-like polypeptide (ELP) and a rare earth element (REE) binding domain are connected directly or by a linker, and such peptides are included in the scope of the present invention regardless of the order of connection, so far as these are connected.

One of X and Y is an amino acid sequence of the elastin-like polypeptide and the other is a rare earth element binding domain (SEQ ID NO: 2).

The elastin-like polypeptide (ELP) is a thermo-responsive biopolymer including a repeating amino acid sequence, also called an elastin-based polypeptide (EBP), which is a terminology widely used in the technical field of the present invention.

ELP has Val-Pro-(Gly or Ala)-Xₐₐ-Gly (SEQ ID NO: 19 or SEQ ID NO: 20) ([VPGXG (SEQ ID NO: 19)] or [VPAXG (SEQ ID NO: 20)]), which is a repeat unit of a pentapeptide as a peptide composed of 5 amino acids. The Xₐₐ(X) is a guest residue and may be any amino acid except for proline. According to the sequence of repeat units, it may be classified into two types of ELPs, wherein one is an ELP having elasticity with a sequence of Val-Pro-Gly-Xₐₐ-Gly (SEQ ID NO: 19), and the other is an ELP having plasticity with a sequence of Val-Pro-Ala-Xₐₐ-Gly (SEQ ID NO: 20).

ELP undergoes a reversible phase transition at the lower critical solution temperature (LCST), also referred to as the transition temperature (Tₜ). Specifically, ELP is highly water soluble at a temperature of lower than Tₜ, but becomes insoluble at a temperature of more than Tₜ, forms aggregates, and exhibits fully reversible LCST behavior over several cycles of cooling and heating. The aggregate may be a coacervate.

In general, the physicochemical properties of ELPs are mostly controlled using a combination of Val-Pro-(Gly or Ala)-Xₐₐ-Gly (SEQ ID NO: 19 or SEQ ID NO: 20), which is a pentapeptide repeat unit. As described below, the transition temperature of ELP may also be adjusted using the combination of the repeat units.

The transition temperature of ELP depends on its amino acid sequence and molecular weight. Regarding the correlation between ELP sequence and Tₜ, many studies were performed by Urry et al. (Urry D. W., Luan C.-H., Parker T. M., Gowda D. C., Parasad K. U., Reid M. C., and Safavy A. 1991. Temperature of polypeptide inverse temperature transition depends on mean residue hydrophobicity. J. Am. Chem. Soc. 113: 4346-4348). Urry et al. found that, in the pentapeptide of Val-Pro-Gly-Val-Gly (SEQ ID NO: 21), when the “guest residue” as the 4th amino acid is substituted with a residue more hydrophilic than Val, Tₜ is increased compared to the original sequence, whereas substitution of the guest residue with more hydrophobic residue decreased the Tₜ than the original sequence. In other words, it was found that ELP having hydrophilic amino acids as guest residues had a relatively high Tₜ, and ELP having hydrophobic amino acids as guest residues had a relatively low Tₜ. Through this finding, it was possible to prepare an ELP having a specific Tₜ by determining which amino acid to use as a guest residue in the ELP sequence and changing the composition ratio of the guest residue (Protein-Protein Interactions: A Molecular Cloning Manual, 2002, Cold Spring Harbor Laboratory Press, Chapter 18. pp. 329-343).

Therefore, those skilled in the art can prepare ELP by introducing various guest residues Xₐₐ into the pentapeptide of Val-Pro-(Gly or Ala)-Xₐₐ-Gly (SEQ ID NO: 19 or SEQ ID NO: 20), using known techniques, and control the transition temperature by changing the guest residue composition ratio.

In one embodiment, the elastin-like polypeptide may consist of the following sequence:

| | SSG |
|---|---|
| (SEQ ID NO: 25) | |
| | (GVGVP) |
| | pGV |
| (SEQ ID NO: 25) | |
| | (GVGVP) |
| | WP |
| | (wherein p is an integer |
| | of 130 to 170). |

In the formula according to one embodiment, p is the repeating number of repeat units of the elastin-like polypeptide, specifically, p may be, for example, an integer of 130 to 170, an integer of 135 to 165, an integer of 140 to 160, or an integer of 145 to 155.

In one embodiment, the elastin-like polypeptide may consist of the amino acid sequence of SEQ ID NO: 1.

The amino acid sequence of SEQ ID NO: 2 is a sequence of a rare earth element (REE) binding domain.

The rare earth element binding domain may be a lanmodulin (LanM) protein or a part thereof. The amino acid sequence of LanM may include four similar EF-hand motifs. The EF-hand motif may include a helix-loop-helix structure very similar to the thumb and forefinger of the human hand.

LanM binds (adsorbs) to lanthanide, a kind of rare earth element, and the metal bound to LanM may be desorbed by decreasing the pH to less than 3 or adding a chelate such as EDTA.

The amino acid sequence of SEQ ID NO: 2 may consist of a sequence excluding the sequence corresponding to the signal peptide from the amino acid sequence of LanM.

As described above, as long as the amino acid sequence of the elastin-like polypeptide is linked to the amino acid sequence of SEQ ID NO: 2, it may be included in the scope of the present invention regardless of the linking order. For example, the peptide of the present invention may include the amino acid sequence of the elastin-like polypeptide and the amino acid sequence of SEQ ID NO: 2; or the amino acid sequence of SEQ ID NO: 2 and the amino acid sequence of an elastin-like polypeptide, in the order from N-terminus to C-terminus.

Further, the amino acid sequence of the elastin-like polypeptide and the amino acid sequence of SEQ ID NO: 2 may be directly linked, and in this case, n may be 0.

The amino acid sequence of the elastin-like polypeptide and the amino acid sequence of SEQ ID NO: 2 may be connected through a linker, and in this case, n may be 1. For example, the peptide of the present invention may include the amino acid sequence of the elastin-like polypeptide, the linker sequence (L) and the amino acid sequence of SEQ ID NO: 2; or the amino acid sequence of SEQ ID NO: 2, the linker sequence (L), and the amino acid sequence of the elastin-like polypeptide, in the order from N-terminus to C-terminus.

In order to minimize degradation after expression when a fusion peptide or fusion protein is expressed in a microorganism (E. coli), the linker is inserted between the peptides or proteins to be fused. As described above, the peptide of the present invention may not include the linker.

When the peptide of the present invention includes a linker, any linker known in the art may be used without limitation. Accordingly, the L is specifically GS, GSGSGSGSGSGSGSGS (SEQ ID NO: 3), GSGSGSGSGSGSGS (SEQ ID NO: 13), GGG, GGGGS (SEQ ID NO: 14), GSGSGS (SEQ ID NO: 15), GGSGG (SEQ ID NO: 16), GGSGGGGG (SEQ ID NO: 17) or GGSGGSGGSGG (SEQ ID NO: 18), but it is not limited thereto.

The peptide of the present invention may further include a tag for peptide purification at the N-terminus or C-terminus. The tag is irrelevant to the function of the peptide of the present invention, and is for purification after preparation of the peptide. As long as the tag is known in the art, it can be used without limitation, and may be, for example, His-tag, Myc tag, HA tag, GST tag, or FLAG tag. When the tag is a His-tag, the tag may be HHHHHH (SEQ ID NO: 22), HHHHHHH (SEQ ID NO: 23) or HHHHHHHH (SEQ ID NO: 24).

The peptide of the present invention may further include methionine (M) for expression in microorganisms. The M is irrelevant to the function of the peptide of the present invention, and the M is generally the first entering amino acid to start translation in E. coli.

In one embodiment, the peptide of the present invention may consist of the following sequence:

(M)m-X-(L)n-Y
(wherein M is methionine, m is 0 or 1, one of X and Y is the amino acid sequence of an elastin-like polypeptide and the other is the amino acid sequence of SEQ ID NO: 2, L is a linker sequence, and n is 0 or 1).

In one embodiment, the peptide of the present invention may consist of the following sequence:

(M)m-(H)k-X-(L)n-Y
(wherein M is methionine, m is 0 or 1, H is histidine, k is an integer of 6 to 8, one of X and Y is the amino acid sequence of the elastin-like polypeptide and the other is the amino acid sequence of SEQ ID NO: 2, L is a linker sequence, and n is 0 or 1).

In one embodiment, the peptide of the present invention may consist of the following sequence:

(M)m-X-(L)n-Y—(H)k
(wherein M is methionine, m is 0 or 1, H is histidine, k is an integer of 6 to 8, one of X and Y is the amino acid sequence of the elastin-like polypeptide and the other is the amino acid sequence of SEQ ID NO: 2, L is a linker sequence, and n is 0 or 1).

In one embodiment, the peptide of the present invention may consist of the sequence of SEQ ID NO: 4.

The peptide of the present invention may include: elastin-like peptides that are linked to each other directly or through a linker and exhibit a reversible phase transition through temperature control; and a domain (SEQ ID NO: 2) strongly adsorbing rare earth elements, such that it is possible to recover the rare earth elements in a high yield and repeatedly use the same through temperature or pH control. Further, since the peptide of the present invention is a kind of biosorbent, it is naturally decomposed and thus the problem of environmental pollution can be minimized.

The peptide of the present invention may be synthesized or produced by transforming a gene (sequence of SEQ ID NO: 5) expressing the peptide into a microorganism.

Further, the present invention provides a composition including the peptide of the present invention.

The composition of the present invention may be a composition for recovering rare earth elements.

As described above, the peptide of the present invention may include an elastin-like peptide and a rare earth element binding domain (SEQ ID NO: 2) linked to each other directly or through a linker.

The present inventors have confirmed that the elastin-like peptide and the rare earth element binding domain included in the peptide of the present invention exhibit a reversible phase transition behavior even when connected to each other, and present characteristics of adsorbing rare earth elements in high yield. Further, the present inventors have confirmed that the peptide of the present invention could selectively recover only rare earth elements from a mixture (artificially prepared mixture or steel slag leachate, etc.) containing both non-rare earth elements and rare earth elements. Therefore, the peptide of the present invention can be used for rare earth element recovery.

The rare earth elements (REEs) to be recovered may be at least one selected from the group consisting of scandium (Sc), yttrium (Y), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb) and lutetium (Lu).

Recovery refers to taking out and collecting materials that have been used once for treatment or operation.

By adding the composition of the present invention to a sample including a rare earth element, the rare earth element may be adsorbed to a peptide included in the composition. Thereafter, the rare earth element adsorbed on the peptide may be desorbed and separated through temperature and pH control in order to recover the rare earth element from the sample. Details are the same as those of the method for recovering rare earth elements, which will be described below.

Further, the present invention provides a method for recovering rare earth elements using the peptide of the present invention.

The rare earth element recovery method of the present invention may include adding the peptide to a sample including the rare earth element to adsorb the rare earth element to the peptide.

When the peptide of the present invention is added to a sample including a rare earth element, the rare earth element in the sample is adsorbed to the rare earth element binding domain included in the peptide.

The step of adsorbing the rare earth element to the peptide may be performed at a temperature less than the transition temperature of the peptide and a pH greater than 3 to 6. Under the above temperature condition, the peptide is water soluble and does not form an aggregate, while the rare earth element remains bound to the rare earth element binding domain in the peptide under the above pH condition. The transition temperature is different depending on the elastin-like polypeptide included in the peptide of the present invention. Further, as described above, the transition temperature may be appropriately adjusted by those skilled in the art. For example, when the peptide of the present invention includes the elastin-like polypeptide of SEQ ID NO: 1, the temperature below the transition temperature may be, for example, less than 30° C., less than 20° C., less than 15° C., less than 10° C. or less than 5° C. Otherwise, the temperature may be greater than 0° C. and less than 30° C., greater than 0° C. and less than 20° C., greater than 0° C. and less than 15° C., greater than 0° C. and less than 10° C., or greater than 0° C. and less than 5° C.

The sample is not limited as long as it includes rare earth elements. For example, the sample may be industrial waste, specifically steel slag, fly ash, electronic waste and the like.

The sample may further include non-rare earth elements (non-REEs). The non-rare earth element may be copper (Cu), zinc (Zn), magnesium (Mg) or calcium (Ca) or the like.

The method for recovering rare earth elements of the present invention may include: adjusting the sample added with the peptide to a temperature higher than the transition temperature of the peptide so as to form aggregates of the peptide to which the rare earth elements are adsorbed; centrifuging the sample to obtain pellets including the aggregates of peptide to which the rare earth elements are adsorbed; adjusting the pellets to a temperature less than the transition temperature of the peptide and a pH of less than 3 thus to solubilize the aggregates of the peptide adsorbed with the rare earth elements and desorb the rare earth elements from the peptide; forming aggregates of the peptide by adjusting the temperature of the mixture including the desorbed rare earth elements and the solubilized peptide to a temperature higher than the transition temperature of the peptide; and centrifuging the mixture and recovering the desorbed rare earth elements from the obtained supernatant.

When the sample added with the peptide is adjusted to a temperature higher than the transition temperature of the peptide, aggregates of the peptide adsorbed with the rare earth elements may be formed. The transition temperature is different depending on the elastin-like polypeptide included in the peptide of the present invention, and as described above, the transition temperature may be appropriately adjusted by those skilled in the art. For example, when the peptide of the present invention includes the elastin-like polypeptide of SEQ ID NO: 1, the temperature above the transition temperature may be, for example, greater than 30° C., greater than 32° C., greater than 33° C., greater than 34° C., greater than 35° C., or greater than 36° C. Otherwise, the temperature may be greater than 30° C. and less than 100° C., greater than 32° C. and less than 100° C., greater than 33° C. and less than 100° C., greater than 34° C. and less than 100° C., greater than 35° C. and less than 100° C., or greater than 36° C. and less than 100° C.

As described above, this is because the elastin-like polypeptide included in the peptide of the present invention undergoes a reversible phase transition at the transition temperature and becomes insoluble aggregates at a temperature below the transition temperature.

The aggregate may be a coacervate.

Since the aggregate is insoluble, the sample may be centrifuged to obtain pellets including the aggregates of the peptide adsorbed with the rare earth elements. A supernatant obtained by centrifuging the sample may contain non-rare earth elements in the sample. The supernatant containing the non-rare earth elements may be discarded.

When the obtained pellets are adjusted to a temperature less than the transition temperature of the peptide and a pH of less than 3, the aggregates of the peptide to which the rare earth elements are adsorbed can be solubilized and the rare earth elements can be desorbed from the peptide.

As described above, this is because the elastin-like polypeptide included in the peptide of the present invention becomes water-soluble at a temperature above the transition temperature, and the rare earth element binding domain included in the peptide desorbs the rare-earth metal bound thereto at a pH of less than 3.

Thereafter, the mixture including the desorbed rare earth element and the solubilized peptide is adjusted to a temperature higher than the transition temperature of the peptide to form an aggregate of the peptide. In addition, after NaCl is further added to the mixture, the temperature of the mixture may be adjusted to form an aggregate of the peptide.

The desorbed rare earth elements may be recovered from the supernatant obtained by centrifuging the mixture. The pellets obtained by centrifuging the mixture may contain aggregates of the peptides in the mixture.

As described above, if using the peptide of the present invention, only rare earth elements can be selectively recovered from the sample including the rare earth elements only by controlling temperature and pH.

The method for recovering rare earth elements of the present invention may further include: recovering the aggregates of the peptide from the pellets obtained by centrifuging the mixture; and adjusting the recovered aggregates of the peptide to a temperature lower than the transition temperature of the peptide and then solubilizing the aggregates. The temperature below the transition temperature is as described above.

By solubilizing the aggregates, the peptide of the present invention may be obtained in a water-soluble form. The peptide obtained by the above steps may be reused to recover rare earth elements. Specifically, the peptide obtained by the above steps may be added to a second sample including a rare earth element, and the rare earth element may be adsorbed to the peptide to recover the rare earth element in the second sample.

Since the peptide of the present invention can recover rare earth elements in high yield even through several rare earth element adsorption/desorption processes and inverse-phase transition processes, the peptide can be repeatedly used by repeating the above-described steps several times.

Hereinafter, the present invention will be described in detail with reference to the following examples.

### Example

### Experimental Materials and Methods

### 1. Compound

Unless otherwise specified, chemical reagents were purchased from Sigma-Aldrich (Saint Louis, MO, USA). Xylenol orange (XO) tetrasodium salt was purchased from Alfa Aesar (Ward Hill, MA, USA). Restriction enzymes BamHI, HindIlI and SalI were obtained from NEB (Ipswich, MA, USA). pET-24a-ELP [V-150] was purchased from Addgene (ID: 67015) (Watertown, MA, USA). All DNA and primers used for cloning were synthesized by Macrogen, Inc (Seoul, South Korea).

### 2. Plasmid Construction for RELP Expression

To construct the bacterial expression of RELP (REE-selective ELP, Rare earth elements-selective Elastin-like polypeptide), pET-24a-ELP [V150] was used as a template. The amino acid and DNA sequences of ELP[V150] were obtained from Addgene (Tables 1 and 2). The LanM gene was inverse translated from the LanM amino acid sequence (UniProtKB C5B164) (Table 1). The codons of the LanM gene were optimized for expression in E. coli using the Codon Optimization Tool (NovoPro Bioscience's ExpOptimizer web server). Based on the codon-optimized gene sequence of LanM (Table 2), a LanM gene containing recognition sites for both BamHI and HindIII was requested to Macrogen for synthesis, which was sub-cloned at the BamHI and HindIII sites of pET-24a-ELP [V150] to construct pET-24a-ELP[V150-LanM]. Next, a duplex oligomer containing recognition sites for both BamHI and Sail (FW: 5′-gatccggctctggltctggltctggtagcggttctggctctggttctgctccaaccacgaccacaaagg-3′, SEQ ID NO: 6; RV: 5′-tcgacctttgtggtcgtggttggagcagaaccagagccagaaccgctacc agaaccagaaccagagccg-3′, SEQ ID NO: 7) was synthesized for removal of the signal peptide (MAFRLSSAVLLAALVAAPAYA, SEQ ID NO: 8) and insertion of a flexible linker (GSGSGSGSGSGSGSGS, SEQ ID NO: 3). Finally, a duplex oligomer containing recognition sites for both BamHI and SalI was subcloned into the BamHI and SalI sites of pET-24a-ELP [V150-LanM] to construct pET-24a-RELP. The amino acid and DNA sequences of ELP[V150-LanM] and RELP are described in Tables 1 and 2.

Table 1 below shows the amino acid sequences and molecular weights of LanM, ELP[V150], ELP[V150]-LanM and RELP. In Table 1 below, the underlined sequences represent signal peptides, and the bold sequences represent linkers.

TABLE 1

| | | Molecular |
|---|---|---|
| | | weight |
| Content | Amino acid sequence | (kDa) |
| LanM | MAFRLSSAVLLAALVAAPAYAAPTTTTKVDIAAFDPDKDG | 13.8 |
| | TIDLKEALAAGSAAFDKLDPDKDGTLDAKELKGRVSEADL | |
| | KKLDPDNDGTLDKKEYLAAVEAQFKAANPDNDGTIDAREL | |
| | ASPAGSALVNLIR (SEQ ID NO: 9) | |
| ELP[V150] | MHHHHHHSSGGVGVPGVGVPGVGVPGVGVPGVGVPGVGVP | 63.1 |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVG | |
| | VPWP (SEQ ID NO: 10) | |
| ELP[V150]- | MHHHHHHSSGGVGVPGVGVPGVGVPGVGVPGVGVPGVGVP | 77.0 |
| LanM | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVG | |
| | VPWPGSMAFRLSSAVLLAALVAAPAYAAPTTTTKVDIAAF | |
| | DPDKDGTIDLKEALAAGSAAFDKLDPDKDGTLDAKELKGR | |
| | VSEADLKKLDPDNDGTLDKKEYLAAVEAQFKAANPDNDGT | |
| | IDARELASPAGSALVNLIR (SEQ ID NO: 11) | |
| RELP | MHHHHHHSSGGVGVPGVGVPGVGVPGVGVPGVGVPGVGVP | 76.0 |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVP | |
| | GVGVPGVGVPGVGVPGVGVPGVGVPGVGVPGVGVPGVGVG | |
| | VPWPGSGSGSGSGSGSGSGSAPTTTTKVDIAAFDPDKDGT | |
| | IDLKEALAAGSAAFDKLDPDKDGTLDAKELKGRVSEADLK | |
| | KLDPDNDGTLDKKEYLAAVEAQFKAANPDNDGTIDARELA | |
| | SPAGSALVNLIR (SEQ ID NO: 4) | |

Table 2 below shows the DNA sequences of LanM and RELP. In Table 2 below, the underlined sequences represent signal peptides, while the bold sequences represent linkers.

TABLE 2

| Con- | |
|---|---|
| tent | DNA sequence |
| LanM | ATGGCGTTTCGCCTTTCAAGTGCCGTCCTGTTAGCTGCCCTTGTT |
| | GCCGCACCAGCATACGCGGCTCCAACCACGACCACAAAGGTCGAC |
| | ATTGCCGCTTTTGACCCCGATAAAGATGGAACGATCGATCTGAAG |
| | GAAGCACTTGCTGCCGGATCAGCCGCGTTCGACAAGCTGGACCCA |
| | GATAAGGACGGAACCCTGGACGCGAAAGAACTGAAAGGTCGTGTC |
| | TCAGAGGCAGACTTAAAAAAACTTGATCCTGACAATGACGGAACC |
| | TTAGATAAAAAGGAATACCTTGCCGCTGTCGAAGCACAATTTAAA |
| | GCGGCGAATCCAGATAATGACGGAACGATTGATGCTCGTGAATTG |
| | GCCTCACCTGCGGGGTCCGCGTTGGTCAACTTAATTCGT (SEQ |
| | ID NO: 12) |
| RELP | ATGCATCATCATCATCATCACAGTTCAGGGGGTGTAGGTGTCCCT |
| | GGAGTAGGCGTTCCAGGCGTCGGGGTACCTGGCGTGGGTGTTCCC |
| | GGCGTTGGAGTCCCGGGTGTAGGAGTCCCCGGTGTTGGTGTACCT |
| | GGAGTGGGAGTACCGGGAGTAGGAGTACCAGGAGTGGGGGTTCCT |
| | GGTGTGGGAGTCCCAGGAGTCGGTGTACCAGGGGTAGGGGTACCG |
| | GGCGTAGGTGTGCCAGGAGTTGGGGTGCCTGGAGTCGGAGTCCCT |
| | GGTGTCGGAGTACCTGGTGTTGGAGTACCCGGAGTTGGTGTTCCT |
| | GGAGTTGGAGTTCCGGGGGTAGGAGTTCCAGGAGTAGGGGTCCCA |
| | GGTGTTGGCGTACCAGGTGTGGGCGTGCCCGGGGTCGGCGTCCCT |
| | GGGGTAGGTGTTCCAGGTGTAGGGGTTCCAGGGGTGGGAGTTCCT |
| | GGGGTTGGAGTGCCTGGTGTAGGTGTCCCAGGAGTAGGTGTCCCT |
| | GGAGTAGGCGTTCCAGGCGTCGGGGTACCTGGCGTGGGTGTTCCC |
| | GGCGTTGGAGTCCCGGGTGTAGGAGTCCCCGGTGTTGGTGTACCT |
| | GGAGTGGGAGTACCGGGAGTAGGAGTACCAGGAGTGGGGGTTCCT |
| | GGTGTGGGAGTCCCAGGAGTCGGTGTACCAGGGGTAGGGGTACCG |
| | GGCGTAGGTGTGCCAGGAGTTGGGGTGCCTGGAGTCGGAGTCCCT |
| | GGTGTCGGAGTACCTGGTGTTGGAGTACCCGGAGTTGGTGTTCCT |
| | GGAGTTGGAGTTCCGGGGGTAGGAGTTCCAGGAGTAGGGGTCCCA |
| | GGTGTTGGCGTACCAGGTGTGGGCGTGCCCGGGGTCGGCGTCCCT |
| | GGGGTAGGTGTTCCAGGTGTAGGGGTTCCAGGGGTGGGAGTTCCT |
| | GGGGTTGGAGTGCCTGGTGTAGGTGTTCCAGGTGTAGGTGTCCCT |
| | GGAGTAGGCGTTCCAGGCGTCGGGGTACCTGGCGTGGGTGTTCCC |
| | GGCGTTGGAGTCCCGGGTGTAGGAGTCCCCGGTGTTGGTGTACCT |
| | GGAGTGGGAGTACCGGGAGTAGGAGTACCAGGAGTGGGGGTTCCT |
| | GGTGTGGGAGTCCCAGGAGTCGGTGTACCAGGGGTAGGGGTACCG |
| | GGCGTAGGTGTGCCAGGAGTTGGGGTGCCTGGAGTCGGAGTCCCT |
| | GGTGTCGGAGTACCTGGTGTTGGAGTACCCGGAGTTGGTGTTCCT |
| | GGAGTTGGAGTTCCGGGGGTAGGAGTTCCAGGAGTAGGGGTCCCA |
| | GGTGTTGGCGTACCAGGTGTGGGCGTGCCCGGGGTCGGCGTCCCT |
| | GGGGTAGGTGTTCCAGGTGTAGGGGTTCCAGGGGTGGGAGTTCCT |
| | GGGGTTGGAGTGCCTGGTGTAGGTGTGCCGGGAGTAGGTGTCCCT |
| | GGAGTAGGCGTTCCAGGCGTCGGGGTACCTGGCGTGGGTGTTCCC |
| | GGCGTTGGAGTCCCGGGTGTAGGAGTCCCCGGTGTTGGTGTACCT |
| | GGAGTGGGAGTACCGGGAGTAGGAGTACCAGGAGTGGGGGTTCCT |
| | GGTGTGGGAGTCCCAGGAGTCGGTGTACCAGGGGTAGGGGTACCG |
| | GGCGTAGGTGTGCCAGGAGTTGGGGTGCCTGGAGTCGGAGTCCCT |
| | GGTGTCGGAGTACCTGGTGTTGGAGTACCCGGAGTTGGTGTTCCT |
| | GGAGTTGGAGTTCCGGGGGTAGGAGTTCCAGGAGTAGGGGTCCCA |
| | GGTGTTGGCGTACCAGGTGTGGGCGTGCCCGGGGTCGGCGTCCCT |
| | GGGGTAGGTGTTCCAGGTGTAGGGGTTCCAGGGGTGGGAGTTCCT |
| | GGGGTTGGAGTGCCTGGTGTAGGTGTTCCTGGGGTAGGTGTCCCT |
| | GGAGTAGGCGTTCCAGGCGTCGGGGTACCTGGCGTGGGTGTTCCC |
| | GGCGTTGGAGTCCCGGGTGTAGGAGTCCCCGGTGTTGGTGTACCT |
| | GGAGTGGGAGTACCGGGAGTAGGAGTACCAGGAGTGGGGGTTCCT |
| | GGTGTGGGAGTCCCAGGAGTCGGTGTACCAGGGGTAGGGGTACCG |
| | GGCGTAGGTGTGCCAGGAGTTGGGGTGCCTGGAGTCGGAGTCCCT |
| | GGTGTCGGAGTACCTGGTGTTGGAGTACCCGGAGTTGGTGTTCCT |
| | GGAGTTGGAGTTCCGGGGGTAGGAGTTCCAGGAGTAGGGGTCCCA |
| | GGTGTTGGCGTACCAGGTGTGGGCGTGCCCGGGGTCGGCGTCCCT |
| | GGGGTAGGTGTTCCAGGTGTAGGGGTTCCAGGGGTGGGAGTTCCT |
| | GGGGTTGGAGTGCCTGGTGTAGGTGTTGGAGTTCCCTGGCCGGGA |
| | TCCGGCTCTGGTTCTGGTTCTGGTAGCGGTTCTGGCTCTGGTTCT |
| | GCTCCAACCACGACCACAAAGGTCGACATTGCCGCTTTTGACCCC |
| | GATAAAGATGGAACGATCGATCTGAAGGAAGCACTTGCTGCCGGA |
| | TCAGCCGCGTTCGACAAGCTGGACCCAGATAAGGACGGAACCCTG |
| | GACGCGAAAGAACTGAAAGGTCGTGTCTCAGAGGCAGACTTAAAA |
| | AAACTTGATCCTGACAATGACGGAACCTTAGATAAAAAGGAATAC |
| | CTTGCCGCTGTCGAAGCACAATTTAAAGCGGCGAATCCAGATAAT |
| | GACGGAACGATTGATGCTCGTGAATTGGCCTCACCTGCGGGGTCC |
| | GCGTTGGTCAACTTAATTCGTTAA (SEQ ID NO: 5) |

### 3. Protein Expression and Purification

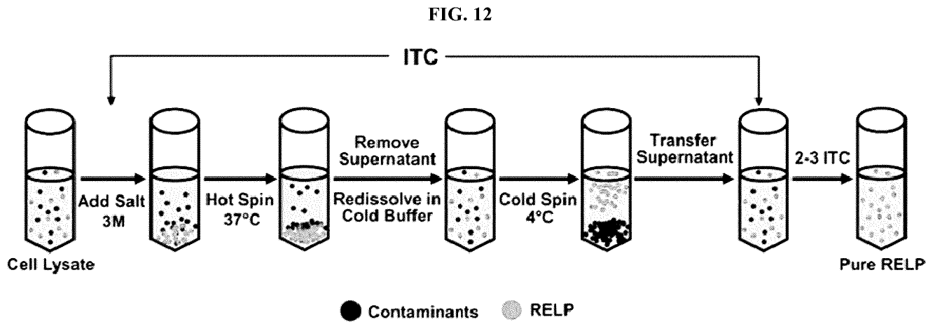

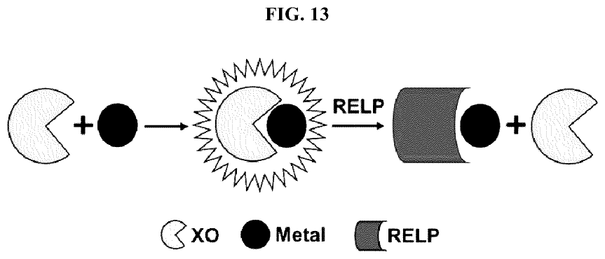

For expression of ELP and RELP proteins, plasmids encoding ELP (pET-24a-ELP[V150]) and RELP (pET-24a-RELP), respectively, were introduced into chemically competent E. coli BL21(DE3) cells. The cells were plated on 1% agar plates with 100 μg/mL kanamycin and incubated overnight at 37° C. Single colonies of transformed cells were pre-cultured in 2×YT medium containing 100 μg/mL kanamycin at 220 rpm and 37° C. for 12 hours. After incubation, the cultured cells were inoculated into fresh 2×YT medium at a 1:100 (v/v) dilution ratio and incubated until the optical density at 600 nm reached 0.5. To induce ELP species, 1 mM of isopropyl β-d-1-thiogalactopyranoside (IPTG) was added to the culture medium and cultured for 8 hours. Cells were pelleted at 5,000 rpm for 5 minutes and stored at −80° C. until further use. For protein purification, the cell pellets were suspended in phosphate-buffered saline (PBS) buffer (pH 7.4). Samples were placed on ice and subjected to lysis at a cycle of 10 seconds amplitude 30% sonication and 20 seconds rest for total 10 minutes. Then, cell debris was removed by centrifugation at 12,000 rpm and 4° C. for 10 minutes. Next, ELP species in the supernatant were purified through an inverse transition cycling (ITC) procedure as described in FIG. 12. The phase transition of ELP species was induced simply by adding 3 M NaCl and then centrifuged at 37° C. and 12,000 rpm for 10 minutes (hot spin). After hot-spinning, the insoluble fraction of the ELP species was dissolved in cold PBS buffer and centrifuged at 12,000 rpm and 4° C. for 10 minutes (cold spin), so as to remove impurities. Each ITC cycle was repeated twice more and the ELP species were finally dissolved in 20 mM MES buffer (pH 5.8) for further characterization.

The concentration of the purified ELP species was determined by measuring the absorbance at 280 nm using a microplate reader (Synergy, BioTek, Winooski, VT, USA) according to the Beer-Lambert law. The absorbance measured at 977 nm was used for path length calibration (BioTek Gen5 sample protocol and experimental guide). The molar-extinction coefficients of ELP and RELP at 280 nm were calculated to be 5,500 and 6,990 M⁻¹ cm⁻¹, respectively, by the following equation: ε₂₈₀=(5500×n_{Trp})+(1490×n_{Tyr})+(125×n_{disulfide bonds}). Protein purity was measured using sodium dodecyl sulfate-polyacrylamide gel electrophoresis (SDS-PAGE). After staining the protein with Coomassie Brilliant Blue R-250, the protein gel was visualized using an imager (ChemiDoc XRS+, Bio-Rad Laboratories, Hercules, CA, USA).

### 4. Characterization of Reversible Phase Transition Behavior

To investigate the effect of LanM fusion on the phase transition of ELP, the phase transition behavior of RELP was characterized and compared with that of ELP. To this end, the inverse transition temperatures (Tₜ) of RELP and ELP were measured. ELP (25 μM) and RELP in 20 mM MES buffer (pH 5.8) were heated from 26° C. to 44° C. at a rate of 1° C./2 min. Then, the change in absorbance at 350 nm (A₃₅₀ nm) was monitored using a microplate reader.

A reversible phase transition behavior was further investigated over several cycles of cooling and heating. First, the sizes of ELP and RELP were measured to investigate the inverse-phase transition. ELP (10 μM) and RELP in 20 mM MES buffer (pH 5.8) were pre-cultured at 37° C. for 10 min each, followed by measuring a size distribution using an electrophoretic light scattering (DLS) system equipped with a diode light source (638 nm) (ELSZ, Otsuka Electronics Osaka, Japan). ELP species were not further diluted for size measurement. Next, the change in the reversible size of ELP and RELP was investigated. ELP (10 μM) and RELP in 20 mM MES buffer were cultured at 10° C. for 20 minutes each, and their sizes were measured as described above. After measuring the sizes at 10° C., the ELP species were recovered and cultured at 37° C. for 20 minutes in order to induce a phase transition. After culturing, the sizes of the ELP species were measured, and such size measurement was further repeated for 2 cycles.

### 5. RELP's Optional REE Binding

To investigate whether RELP can selectively bind to REE, xylenol orange (XO) was used for spectrophotometric determination of REE and non-REE metal ions. The color change of XO was monitored by measuring the absorbance at 575 nm (A_{575 nm}), which is decreased when the metal forms a complex with XO. Experiments were performed using 96-well plates containing 7 μM XO and 0 or 3 μM RELP. Various concentrations (1, 2 and 3 μM) of REE (Tb³⁺, La³⁺, Ce³⁺ and Y³⁺) and various concentrations (2, 4, 6, 8 and 10 μM) of non-REE (Zn²⁺ and Cu²⁺) were used. Experiments were performed in 20 mM MES buffer (pH 5.8). The UV-visible spectrum was obtained between 240 and 800 nm using a microplate reader, and the metal ion concentration was plotted by adjusting A_{575 nm} according to the volume of the solution.

### 6. REE Binding Ability and Stoichiometry of RELP

To investigate the stoichiometry and maximal REE binding ability of RELP, a competition assay was performed using the colorimetric probe, XO, as previously described. Since efficient REE recovery using RELP requires high performance and selectivity for REE, the molar concentration ratio of REE per RELP and selectivity for REE in the presence of non-REE ions (in this case, Ca²⁺ ions) were measured. Titration of XO with REE increases A_{575 nm} due to the formation of a REE-XO complex. However, when Ca²⁺ ions were added, no change in the relative intensity of A_{575 nm} was observed. 96-well plates were prepared with 7 μM XO and 0 or 5 μM RELP. The range of different concentrations of the REE solution is 2 to 20 μM. A_{575 nm} was also scaled according to the volume of the solution and plotted relative to the metal ion concentration. Experiments were implemented in 20 mM MES buffer (pH 5.8). The stoichiometric point at which the binding site of RELP is saturated was estimated based on the metal ion concentration at 10% saturation of XO, followed by determining the same as follows: A=Aᵢₙᵢₜᵢₐₗ+0.1 (A_{final}−Aᵢₙᵢₜᵢₐₗ). In experiments using proteins, pre-mixing of buffer and protein was conducted, and spectra were acquired before adding XO to calculate the protein concentration in the cuvette.

### 7. REE Recovery at Different pH

To test the REE recovery of RELP at various pH conditions (pH 2, 3, 4, 5 and 6), the pH of the 20 mM MES buffer was adjusted accordingly. In recovery experiments, 100 μM RELP was mixed with 100 μM Tb³⁺, and Tb³⁺ was recovered using the above procedure. Concentrations of unbound and recovered metals were measured in the supernatant using ICP-MS at the Central Research Facility (GCRF) facility of the Gwangju Institute of Science and Technology (Agilent 7900, Agilent Technologies, Santa Clara, CA, USA). Samples were diluted in 2% HNO₃ for ICP-MS analysis. ICP-MS results correspond to mean values (n=3). The percentage of metal recovered was calculated relative to the amount of metal initially added.

### 8. Selective Binding and Recovery of REE Using RELP

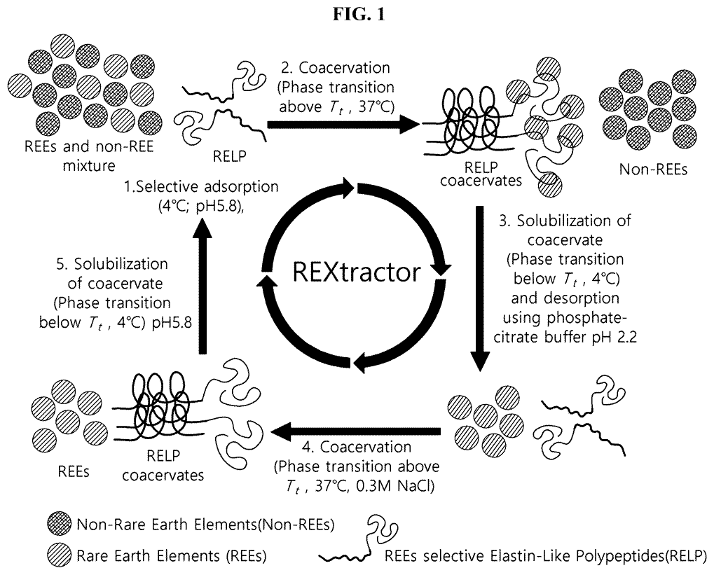

Since non-REE species such as Mg²⁺ and Zn²⁺ are considered as strong competitors, a batch study was performed to investigate the selective binding and recovery of REE by RELP. 100 μM of a mixed equimolar solution of 13 REEs (Y³⁺, La³⁺, Ce³⁺, Pr³⁺, Nd³⁺, Sm³⁺, Eu³⁺, Gd³⁺, Tb³⁺, Ho³⁺, Er³⁺, Tm³⁺ and Lu³⁺), 2 mM of a mixed equimolar solution of Mg²⁺ and Zn²⁺, and 100 μM of RELP were added to 20 nM MES buffer (pH 5.8) while stirring at 4° C. for 30 minutes. The sample was placed on a heating block at 37° C. for 10 minutes and centrifuged at 12,000 rpm and 37° C. for 10 minutes to perform a phase transition above Tₜ for 10 minutes. The supernatant transferred to anew tube was used for unbound metal analysis. Next, RELP coacervates (pellets) were re-solubilized (phase transition below Tₜ) in ice-cold phosphate-citrate buffer (4 mM Na₂HPO₄, 100 mM citric acid, pH 2.2) while stirring at 4° C. for 30 minutes for desorption of bound metals from RELP. In order to separate the RELP and the desorbed metal, 20 μL of 3 M NaCl was added to the sample, followed by a phase transition above Tₜ as described above to obtain the RELP coacervate, and the supernatant was recovered and used for the analysis of the recovered metal (see REE recovery cycle using RELP in FIG. 1). Concentrations of unbound and recovered metal were measured in the supernatant using ICP-MS as described above. The concentration of metal bound to RELP was calculated by subtracting the initial concentration from the unbound concentration.

### 9. Repeated Recovery of REE Using RELP

To evaluate whether RELP can repeatedly recover REE for practical applications, the same RELP was used for multiple cycles for Tb³⁺ recovery. Recovery experiments were performed using the procedure described above (see FIG. 1). In order to determine recovery capacity, 75 μM of RELP was mixed with about 200 μM of Tb³⁺ For subsequent cycles, the RELP coacervate (pellets) obtained after the first cycle was resolubilized in ice-cold 20 mM MES buffer (pH 5.8) containing about 200 μM Tb³⁺ followed by performing maximum 6 cycles at 4° C. for 10 min. For recovery efficiency studies, 100 μM of RELP was mixed with about 50 μM Tb³⁺. Subsequent cycles were repeated for up to 8 cycles. The concentration of recovered REE was determined using ICP-MS as described above.

10. Recovery of REE from Steel Slag Leachate by RELP

Steel slag, specifically basic oxygen furnace (BOF), was provided by a domestic steelmaking company. The particle size (dp) of the steel slag was controlled to be less than 150 μm (avg. 121.2±4.9 μm), and the slag was extracted without separate pretreatment. Hydrochloric acid (HCl, 35%, Daejung Chemicals & Metals Co., LTD., Korea) was used as an extractant. To extract rare earth elements from steel slag, 10 g of slag powder was leached in 80 ml of 2 M HCl solution for 2 hours. The reaction was carried out in a batch reactor system while maintaining the temperature at 25° C. and stirring at 500 rpm. After leaching, the leachate was filtered through 0.45 μm filter paper. The concentration of rare earth elements in the leachate was measured again using ICP-MS. REE recovery from steel slag leachate was performed at a total REE concentration of about 50 μM, which was diluted in 20 mM MES buffer before adding RELP (50 μM). Recovery experiments were performed as described above. The concentration of recovered REE was determined using ICP-MS as described above.

### Experiment Results

### 1. RELP (REE-Selective ELP) Design and Purification

Elastin-like polypeptide (ELP) sequences were designed to achieve the expected Tₜ (transition temperature) above 30° C. to promote aggregation in response to small increase of temperature. Since the composition and chain length of guest residues strongly influence on Tₜ, appropriate changes in sequence and composition may affect the phase behavior. The ELP gene encoding valine (Val) as a guest residue consisted of 150 repeats of the [GVGVP (SEQ ID NO: 25)] amino acid motif with Tₜ of about 30° C. and a molecular weight of about 63 kDa in aqueous solution (ELP[V150]). As rare earth elements (REE)-binding domain (REE-binding domain), Lanmodulin domain (LanM) was fused to the C-terminus of ELP to develop REE-selective ELP (RELP) (Table 1).

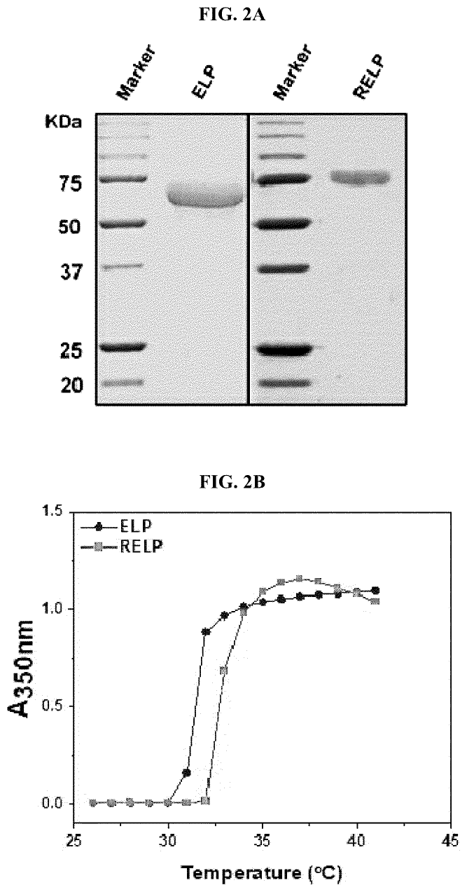

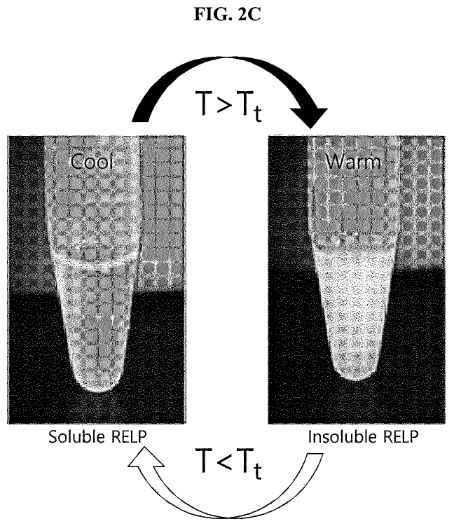

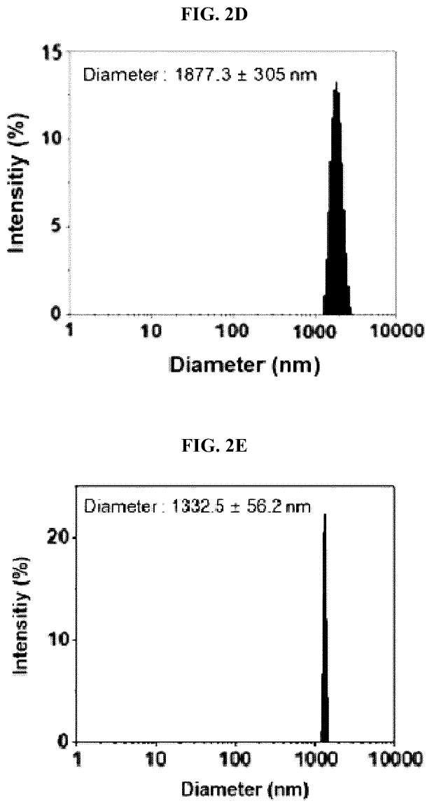

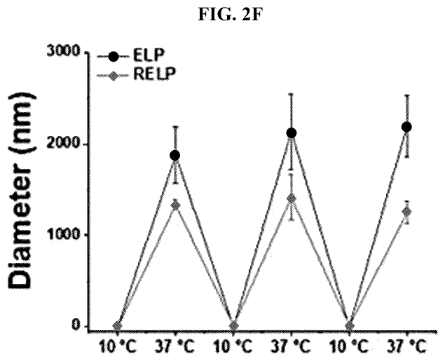

As can be seen from the RELP sequence in Table 1, the LanM signal peptide (MAFRLSSAVLLAALVAAPAYA, SEQ ID NO: 8) was excluded because it was not essential for binding to REE. After expression in E. coli cells, both ELP and RELP were purified through ITC (inverse transition cycling). The theoretical molecular weights of the purified ELP and RELP are about 63 and 75 kDa, respectively. Protein bands corresponding to ELP and RELP were observed in the protein gel, and the purity of each of ELP and RELP was more than 95% (FIG. 2A).

### 2. Phase-Transition Characteristics of RELP

Since the phase transition of ELP can be influenced by length, sequence, structure and fusion partner, the thermal behavior of RELP was characterized and compared with ELP. First, by monitoring the turbidity (A_{350 nm}) of the solution at 350 nm, the transition temperatures (Tₜ) of ELP and RELP were measured. A rapid increase in turbidity for both ELP and RELP was observed as the temperature was increased above the critical point (FIG. 2B), which indicates that coacervate was produced by the sol-gel transition. The value of Tₜ was taken as the temperature at which 50% turbidity occurred, and then used as an index of phase transition. In 20 mM MES buffer (pH 5.8), ELP exhibited a Tₜ value of 30° C., which was increased to 32° C. for RELP. Since the Tₜ of ELP fused with a protein may be affected by the solvent accessible area of the fused protein, the difference in Tₜ between ELP and RELP is expected to be due to different features such as hydrophilic property, exposure of amino acid side chains, etc., between LanM and ELP. However, considering that Tₜ of RELP is slightly larger than Tₜ of ELP, the fusion of LanM did not significantly affect the phase transition properties of RELP. The formation of the RELP coacervate was reversible and the precipitate was completely resolubilized when the solution temperature was reduced below Tₜ (FIG. 2C). Further, both ELP and RELP showed micrometer-scale coacervates at 37° C., as shown by DLS analysis (FIGS. 2D and 2E). In contrast to ELP (FIG. 2D), the RELP coacervate appeared to have a more consistent size distribution because the fused LanM could stabilize the surface between ELP and solution (FIG. 2E). Further, compared to ELP, RELP can maintain reversible phase transition properties during 3 cycles of reversible temperature change (Tₜ or less (10° C.) and Tₜ or more (37° C.)) (FIG. 2F). From these results, the present inventors demonstrated that LanM could be fused into ELP while minimizing perturbation of ELP's thermo-responsive properties. This reversible conversion of RELP is a necessary property for repeatedly using RELP for REE recovery.

### 3. Selectivity of RELP

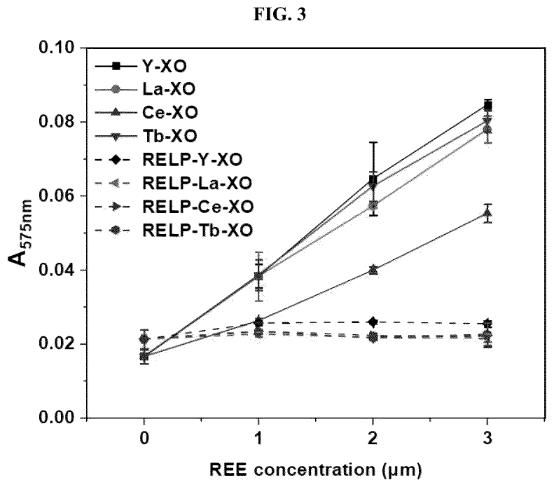

The present inventors evaluated the selective REE-binding properties of RELP based on metal binding experiments by a simple and facile colorimetric dye-displacement assay. Competition between RELP and xylenol orange (XO) enables detection of RELP-metal binding by monitoring a color change of XO. When each REE (Tb³⁺, La³⁺, Ce³⁺ and Y³⁺) was incubated with XO, A_{575 nm} was increased due to the formation of a REE-XO complex (Y—XO, La—XO, Ce—XO and Tb—XO in FIG. 3). When RELP was added to a mixture of XO and REE, A_{575 nm} was decreased due to the formation of the REE-RELP complex and the dissociation of the REE-XO complex (RELP-Y—XO, RELP-La—XO, RELP-Ce—XO and RELP-Tb—XO in FIG. 3). These results indicate that the LanM portion of RELP retains the binding ability of REE.

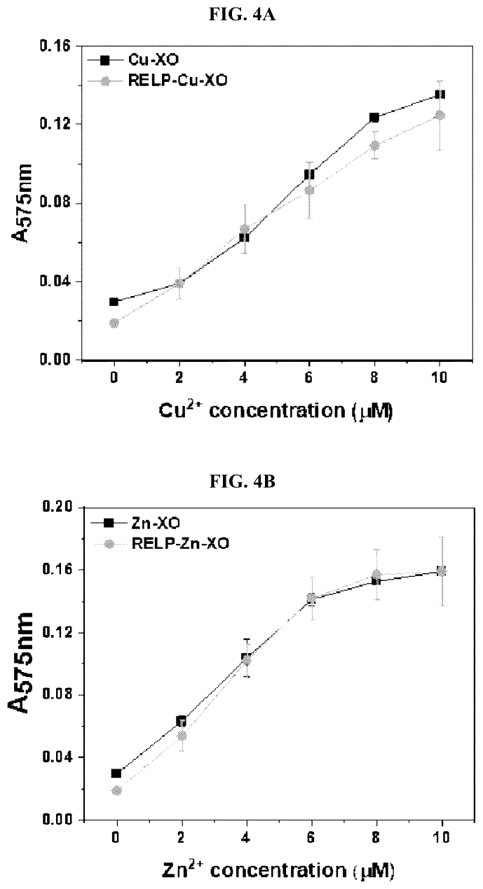

The present inventors confirmed the selective REE-binding of RELP in the presence of non-rare earth elements (non-REEs). Since the concentration of non-REE is higher by tens of times than the concentration of REE from low-grade sources, high selectivity for REE is important in order to substantially recover REE in the presence of non-REE. First, the binding of RELP to Zn²⁺ and Cu²⁺ as competing non-REE ions was investigated. Zn²⁺ and Cu²⁺ were selected as non-REE ions for this experiment since these are present in the high concentration (in the μM range) in environmental media and industrial feedstock. In the absence of RELP, XO formed a 1:1 complex with Cu²⁺ and the relative intensity of A_{575 nm} was increased as the metal concentration was increased. In the presence of RELP, no significant decrease in A_{575 nm} was observed, which indicates that RELP did not bind to Cu²⁺ (FIG. 4A). Similar results were also obtained for Zn²⁺ (FIG. 4B). Considering that the concentrations of Zn²⁺ and Cu²⁺ are higher than the concentrations of REE (Tb³⁺, La³⁺, Ce³⁺, and Y³⁺) in FIG. 3, it can be seen that the affinity is not good. These results show that RELP does not outperform XO for binding to non-REE. LanM maintained the binding ability to REE even in the presence of 1.5 M Mg²⁺, 1.5 M Ca²⁺, 0.4 M Zn²⁺ and 10 mM Cu²⁺.

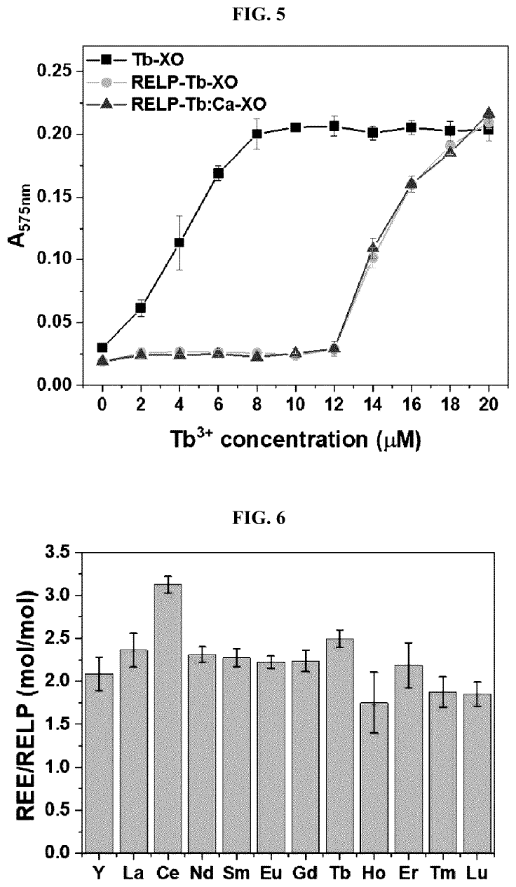

Next, the present inventors investigated the maximal REE-binding ability of RELP by a competition assay in which titration of XO with Tb³⁺ is carried out in the presence or absence of RELP (5.0 μM). The increase in A_{575 nm} due to the formation of the Tb—XO complex observed in the absence of RELP (Tb—XO in FIG. 5) was completely suppressed until Tb³⁺ concentration reached 12 μM in the presence of RELP (RELP-Tb—XO in FIG. 5). The absorbance of XO at 12 μM of Tb³⁺ was taken as a saturation value. The endpoint was considered when A_{575 nm} rose by 10% of the total observed increase (herein at 12 μM Tb³⁺), representing that the Tb³⁺ (REE/RELP) bound to each RELP was 2.5 (FIGS. 5 and 6). In order to confirm the generality, the present inventors conducted the same analysis for other REEs (Y³⁺ La³⁺, Ce³⁺, Nd³⁺, Sm³⁺, Eu³⁺, Gd³⁺, Ho³⁺, Er³⁺ Tm³⁺ and Lu³⁺). As a result, RELP showed similar binding ability to 12 other REEs (FIG. 6).

Further, the present inventors investigated the selectivity of RELP for REE compared to non-REE. Ca²⁺ is generally regarded as a strong competitor of REE ions because Ca²⁺ has an ionic radius (1 Å) similar to that of REE ions and thus is used as a representative non-REE. RELP maintained REE binding affinity and maximum REE binding ability even in the presence of Ca²⁺ (RELP-Tb:Ca—XO in FIG. 5). Overall, these findings demonstrate that LanM fused with ELP retains high affinity and selectivity for REE.

### 4. REE Extraction and Recovery Using RELP

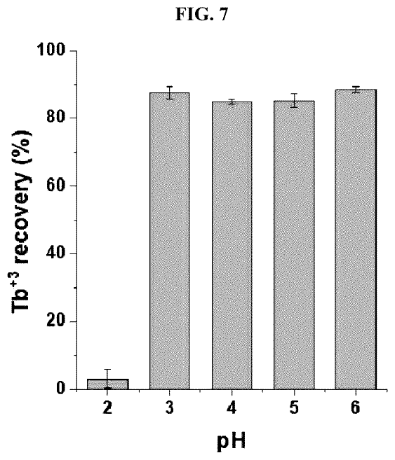

REE extraction usually involves acid leaching methods. Therefore, REE (Tb³⁺) recovery was performed under various acidic conditions (pH 2-6). At higher pH (pH≥6), REE tends to form hydroxide complexes and precipitate, making recovery of REE relatively easier than at pH below 6. The effect of pH on REE recovery using RELP is shown in FIG. 7. At all pH values except pH 2, the REE recovery efficiency was similar to one another. Desorption of REE from RELP was performed in buffer (pH 2.2). These results (FIG. 7) suggest that RELP is particularly suitable for REE extraction from acidic feedstock and the harsh physicochemical conditions commonly encountered in hydrometallurgical processes with low pH value. Further, these results indicate the reusability of RELP and the applicability of RELP in industrial processes by adsorbing and desorbing REE in the pH cycle (FIG. 1).

Since REE coexists with other metals in ore and waste streams, it was investigated whether RELP could selectively extract REE from a mixture of REE and non-REE ions. REE recovery by RELP was attempted in the presence of Mg²⁺ and Zn²⁺ as challengers. These competent elements were chosen because REE coexists predominantly with these metals at the high concentration (in the high μM range) in ore and waste streams. Ca²⁺ was not selected because it interferes with the ICP-MS analysis of REE. A batch study was conducted to investigate the selective REE recovery by RELP using an equimolar solution of 13 REEs and 2 non-REEs as mock waste. The total concentration of non-REE in the mock waste was 20-fold or 300-fold higher than the total concentration of all REEs or the concentration of each REE, respectively. RELP (100 μM) was added to the mock waste, followed by REE recovery using successive phase transitions. As in the experimental materials and methods described above, RELP was incubated at 37° C. for 10 minutes for coacervation and at 4° C. for 30 minutes for resolubilization.

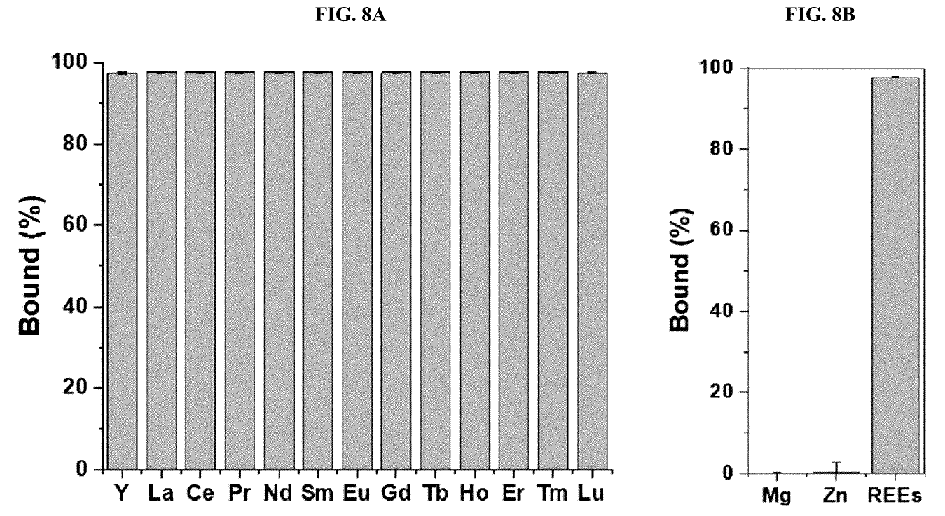

ICP-MS analysis of mock waste after RELP treatment showed that all 13 REEs exhibited similar binding ability to RELP (FIG. 8A), which is consistent with the previously described results (FIG. 6). More than 95% of total REE was bound to RELP (FIG. 8B). RELP was found to hardly bind to two non-REE elements (Mg²⁺ and Zn²⁺) (FIG. 8B). These results clearly indicate that, even in the presence of a metal mixture including excess Mg²⁺⁻ and Zn²⁺ ions, competent metal ions do not significantly affect the amount of REE bound to RELP.

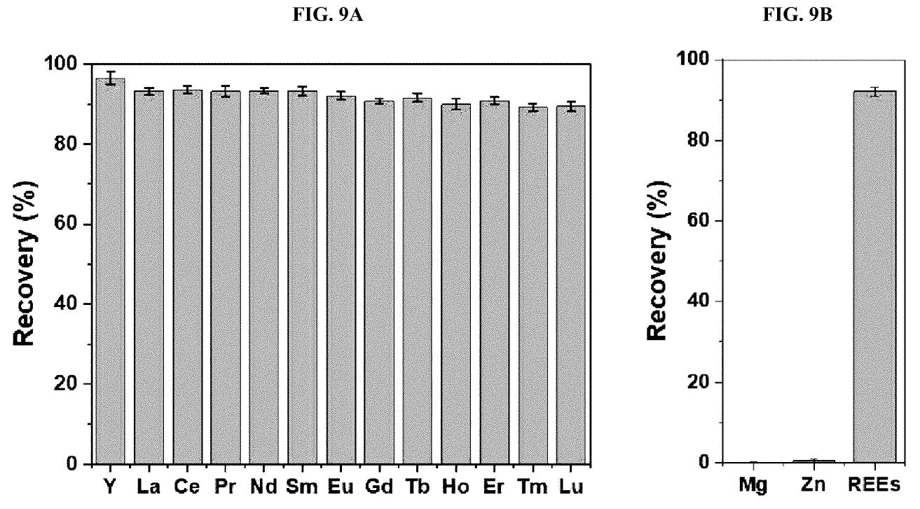

Further, in the presence of non-REE (Mg²⁺ and Zn²⁺), REE recovery efficiency by RELP was investigated. The metal may be desorbed by lowering the pH to 2 using nitric acid/citric acid or by adding a chelating agent such as EDTA, wherein the desorption of REE from RELP in the present example was conducted using a phosphate-citrate buffer (pH 2.2). The percentage of recovered metal was calculated as an amount of metal recovered relative to the amount of metal initially added (FIG. 9A). As the amounts of 13 REEs bound to RELP were similar to one another (FIG. 8A), the recovery yields of all 13 REEs were also similar to one another (FIG. 9A). Total recovery yield of all REEs was greater than 92% (FIG. 9B). The total recovery yield of all non-REE was less than 0.1% (FIG. 9B). Recovery data for non-REE (Mg²⁺ and Zn²⁺) are shown in FIG. 8B. These results indicate that competing metal ions did not affect the REE recovery performance of RELP. Even when a content of the competing ions was high and the REE content was low, RELP selectively recovered REE while leaving non-REEs. Therefore, the phase transition and desorption steps after addition of RELP to a metal mixture containing an excess amount of non-REE demonstrate that a concentrated and purified REE solution can be obtained by selectively recovering REE from a low-purity REE solution.

### 5. RELP Reuse for Repeated Recovery of REE

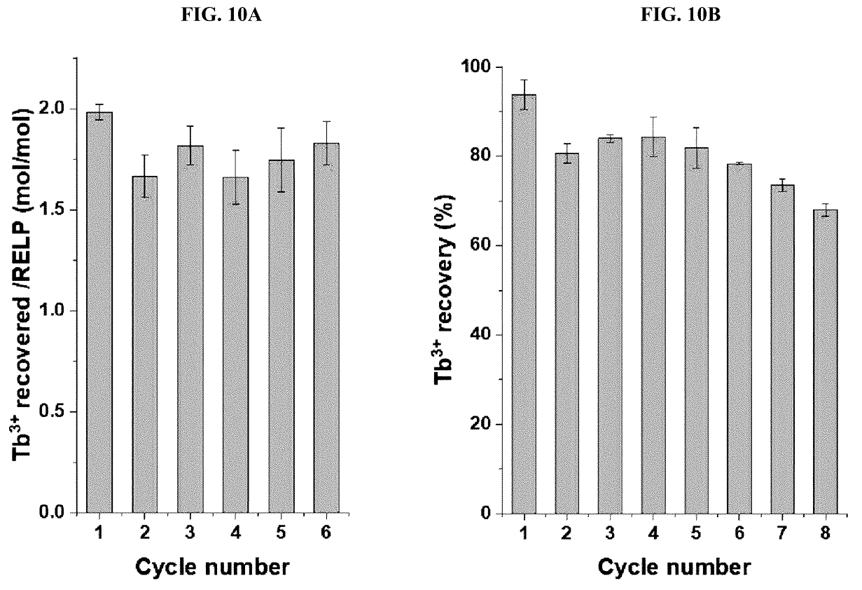

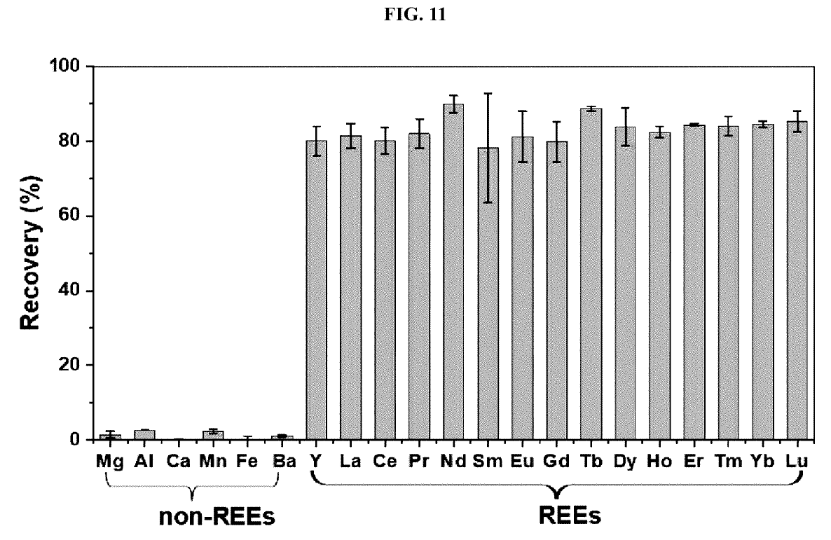

Regeneration of metal binding sites is essential for repeatable use and successful practical applications. Although the viability of the cell-based adsorbent is compromised by acid washing, the RELP of the present example is expected to be more resilient to harsh environmental conditions. To assess the reusability, the REE recovery capacity and efficiency of RELP were evaluated by ICP-MS over several cycles. The REE recovery capacity was determined by calculating the amount of Tb³⁺/RELP (mol/mol) recovered over 6 consecutive cycles. As can be seen in FIG. 10A, the recovery performance of RELP was not significantly decreased even after 6 regeneration cycles (FIG. 1), which indicates a consistent saturation capacity of RELP (REE:RELP stoichiometric ratio of about 2:1) (FIG. 5). This reusability of RELP makes RELP much more economical for large-scale practical use while maintaining the function of RELP. As shown in FIG. 10B, the recovery efficiency of RELP for 50 μM of Tb³⁺ was further investigated for 8 consecutive cycles using the regenerated RELP. ICP-MS analysis of REE concentration showed that RELP exhibited an REE recovery efficiency of 80% or more even up to 5 cycles. These results show that RELP can withstand several rounds of adsorption/desorption and inverse-phase transition up to 5 times with an efficiency of 80% or more.

6. Recovery of REE from Steel Slag Leachate by RELP

A real sample of REE is of practical importance. This generally has higher concentrations of various non-REE ions and very low REE content. Further, real samples are considered as REE's resources. In particular, ferrous metallurgical slag is an abundant industrial waste and a potentially valuable resource for REE. In recent years, there has been a growing interest in value-recovery of steelmaking slag. Important raw materials such as REE are present in significant quantities of slag. Furthermore, the steel industry presents a serious environmental problem by generating a large amount of steel slag as waste and landfill.

REE was repeatedly recovered from steel slag samples via RELP to further demonstrate the feasibility of the present invention for treating real samples. A low-grade leachate (about 0.13 mol % REE, excluding monovalent ions) was prepared from industrial steel slag as a by-product obtained from the steelmaking industry. The steel slag leachate sample contained about 41 mM metal ions, including about 55 μM REE and other metals (8.4 mM Mg, 2.2 mM Al, 28.1 mM Ca, and 2 mM Fe) (Table 3). As expected, the recovery efficiency of various REEs by RELP from steel slag leachate was about 80%. Unlike REE, the recovery efficiency of non-REE by RELP was insignificant as shown in FIG. 11.

Table 3 below shows the initial metal concentration in the steel slag leachate at pH 5.6.

TABLE 3

| Metal | Concentration (mM) |
|---|---|
| Mg | 8369 |
| Al | 2188 |
| Ca | 28120 |
| Mn | 719 |
| Fe | 2070 |
| Ba | 2.026 |
| Y | 2.082 |
| La | 11.77 |
| Ce | 27.61 |
| Pr | 3.088 |
| Nd | 11.07 |
| Sm | 1.202 |
| Eu | 0.190 |
| Gd | 0.461 |
| Tb | 0.046 |
| Dy | 0.210 |
| Ho | 0.038 |
| Er | 0.102 |
| Tm | 0.013 |
| Yb | 0.068 |
| Lu | 0.011 |

### REFERENCE TO AN ELECTRONIC SEQUENCE LISTING

A sequence listing electronically submitted on Jan. 19, 2024 as a XML file named 20240119_LC0742403_TU_SEQ.XML, created on Jan. 19, 2024 and having a size of 29,288 bytes, is incorporated herein by reference in its entirety.

1: A peptide comprising the following sequence:
X-(L)n-Y
wherein one of X and Y is an amino acid sequence of an elastin-like polypeptide, the other is an amino acid sequence of SEQ ID NO: 2, L is a linker sequence, and n is 0 or 1.

2: The peptide according to claim 1, wherein the elastin-like polypeptide consists of the amino acid sequence of SEQ ID NO: 1.

3: The peptide according to claim 1, wherein L is GS or an amino acid sequence of SEQ ID NO: 3.

4: A composition for recovering rare earth elements, comprising the peptide according to claim 1.

5: A method for recovering rare earth elements, the method comprising:
adding the peptide according to claim 1 to a sample including rare earth elements to adsorb the rare earth elements to the peptide.

6: The method according to claim 5, wherein the step of adsorbing the rare earth element to the peptide is performed at a temperature less than a transition temperature of the peptide and a pH greater than 3 to 6.

7: The method according to claim 5, further comprising:
adjusting the sample added with the peptide to a temperature higher than the transition temperature of the peptide to form aggregates of the peptide to which the rare earth elements are adsorbed;
centrifuging the sample to obtain pellets including aggregates of the peptide to which the rare earth elements are adsorbed;
adjusting the pellets to a temperature less than the transition temperature of the peptide and a pH of less than 3 thus to solubilize the aggregates of the peptide adsorbed with the rare earth elements and desorb the rare earth elements from the peptide;
forming the aggregates of the peptide by adjusting the mixture including the desorbed rare earth elements and the solubilized peptide to a temperature higher than the transition temperature of the peptide; and
recovering the desorbed rare earth elements from the supernatant isolated and obtained by centrifuging the mixture.

8: The method according to claim 7, further comprising:
recovering the aggregates of the peptide from the pellets obtained by centrifuging the mixture; and
adjusting the recovered aggregates of the peptide to a temperature less than the transition temperature of the peptide thus to solubilize the aggregates.
